(19) United States
(12) Patent Application Publication
(10) Pub. No.: US 2021/0330350 A1
(43) Pub. Date: Oct. 28, 2021

(54) Trans-Ventricular Introducer Sheath With Intracardiac Extracardiac Stabilization and a Suture Retention System

(71) Applicant: Haven Sheath, LLC, Delafield, WI (US)

(72) Inventors: Scott Koss, Delfield, WI (US); Robert Gessert, Ft. Myers, FL (US)

(21) Appl. No.: 16/858,608

(22) Filed: Apr. 25, 2020

(51) Int. Cl. A61B 17/34; A61B 17/00

(52) U.S. Cl. CPC A61B 17/3403; A61B 17/00234; A61B 17/3417; A61B 2017/1142; A61B 2017/00243; A61B 2017/3486; A61B 17/3498

(57) ABSTRACT

An intra-cardiac introducer device includes a sheath having an inner wall and an outer wall spaced apart from the inner wall to define a sheath space, the sheath defining a distal end and a proximal end and arranged to penetrate cardiac tissue. A hub is connected to the proximal end of the sheath, an intracardiac stabilization balloon is coupled to the distal end of the sheath, and an external stabilization disc is slidingly positioned along the sheath. The cardiac tissue is sandwiched between the intracardiac stabilization balloon and the external stabilization disc to secure the position of the sheath in the cardiac tissue.

### BACKGROUND

During transventricular “beating heart” endovascular thoracic aortic surgery and structural heart surgery, including mitral/aortic/tricuspid/pulmonic valve replacement and suture mediated repair of mitral/tricuspid valves, there is a need for a safe and stable transventricular introducer sheath to minimize blood loss and reduce myocardial injury during insertion and removal of intracardiac/endovascular devices. With suture mediated transventricular structural heart surgery, there are numerous sutures that protrude through the ventriculotomy at the left or right ventricular apical region. The organization of the valve sutures and minimization of instruments on the field (which are at risk for inadvertent removal), along with a stable and hemostatic introducer sheath are critical to the safety and success of the surgery.

### BRIEF SUMMARY

In one construction, an intra-cardiac introducer device includes a sheath having a tubular portion that has an inner wall and an outer wall spaced apart from the inner wall to define an annular space, the sheath defining a distal end and a proximal end and arranged to penetrate cardiac tissue. A hub is connected to the proximal end of the sheath, an intracardiac stabilization balloon is coupled to the distal end of the sheath, and an external stabilization disc is slidingly positioned along the sheath. The cardiac tissue is sandwiched between the intracardiac stabilization balloon and the external stabilization disc to secure the position of the sheath in the cardiac tissue.

In another construction, an intra-cardiac introducer device includes a sheath having a tubular structure that includes an inner wall and an outer wall each extending from a distal end to a proximal end, the inner wall and the outer wall arranged concentrically to define an annular space. A hub is connected to the proximal end of the sheath and a suture retention system is removably coupled to the hub. The suture retention system includes a body, a plurality of slots on the body configured to receive sutures, and an orientation marker configured to identify a position of the sutures relative to a patient's heart. The sutures are selectively disposed at least partially within the annular space.

### BRIEF DESCRIPTION OF THE DRAWINGS

FIG. 1 illustrates an intra-cardiac introducer device.

FIGS. 2A-D illustrate additional views of the intra-cardiac introducer device illustrated in FIG. 1 in different states.

FIG. 3 is a top view of an extra-cardiac stabilization disc.

FIG. 4 is a cross-section view of a valve system.

FIG. 5 is a top view of a first valve of the valve system illustrated in FIG. 4.

FIG. 6 is a perspective view of a second valve of the valve system illustrated in FIG. 4.

FIG. 7 is a top view of a suture retention system connected to the hemostatic hub.

FIG. 8 illustrates a portion of a transventricular“beating heart” suture mediated repair of the mitral valve.

FIG. 9 illustrates a portion of a transventricular “beating heart” suture mediated repair of the mitral valve.

FIG. 10 illustrates a portion of a transventricular “beating heart” suture mediated repair of the mitral valve.

FIG. 11 is a top view of the suture retention system connected to the hemostatic hub used during a portion of a transventricular “beating heart” suture mediated repair of mitral/tricuspid valves.

FIG. 12 is a top view of the suture retention system removed from the hemostatic hub used during a portion of a transventricular “beating heart” suture mediated repair of mitral/tricuspid valves.

FIG. 13 illustrates a further rendering of the intra-cardiac introducer device shown in FIG. 1 being utilized to access the mitral valve.

FIG. 14 illustrates a further rendering of the intra-cardiac introducer device shown in FIG. 1 being utilized to access the aortic valve.

FIG. 15 illustrates a further rendering of the intra-cardiac introducer device shown in FIG. 1 being utilized to access the tricuspid valve.

FIG. 16 illustrates a further rendering of the intra-cardiac introducer device shown in FIG. 1 being utilized to access the pulmonic artery.

FIGS. 17A-B illustrate another intra-cardiac introducer device.

FIG. 18 is an end view of another tubular structure including deployed but unseated sutures.

FIG. 19 is an end view of the tubular structure of FIG. 18 including deployed sutures seated in an annular space.

FIG. 20 is an end view of the tubular structure of FIG. 18 rotated ninety degrees and including deployed but unseated sutures.

FIG. 21 is an end view of the tubular structure of FIG. 19 rotated ninety degrees including deployed sutures seated in an annular space.

FIG. 22 is a top view of the suture retention system connected to the hemostatic hub used during a portion of a transventricular “beating heart” suture mediated repair of mitral/tricuspid valves including the tubular structure of FIGS. 18-21.

### DETAILED DESCRIPTION

It should be understood that the words or phrases used herein should be construed broadly, unless expressly limited. For example, the terms “include,” “comprise,” and “having” as well as derivatives should be interpreted as inclusive without limitation. The singular forms “a”, “an” and “the” are intended to include the plural forms as well, unless the context clearly indicates otherwise. Further, the term “and/or” as used herein refers to and encompasses any and all possible combinations of one or more of the associated listed items.

Also, although terms such as “first”, “second”, “third” and so forth may be used herein to refer to various elements, information, functions, or acts, these elements, information, functions, or acts should not be limited by these terms and no specific order should be implied or interpreted. Rather these numeral adjectives are used to distinguish different elements, information, functions or acts from each other.

The phrase “adjacent to” may mean that an element is relatively near to but not in contact with another element or that the element is in contact with the other element, unless the context clearly indicates otherwise. In addition, in some contexts, “adjacent to” or “near to” and similar phrases simply mean that the element is closer to the other element described. For example, an element described as being “near” a first end of another element having a first end and a second end is simply positioned closer to the first end than to the second end.

Terms of degree such as “about”, approximately, and the like are generally interpreted as being within well-known industrial tolerances for the particular feature. If no such standard exists, terms of degree should be interpreted as meaning plus or minus twenty percent unless another clear standard is provided.

None of the following description should be read as implying that any particular element, step, act, or function is an essential element, which must be included in the claim scope. In addition, while multiple variations, embodiments, and constructions are described, it should be understood that any aspect described with one embodiment, variation, or construction is equally applicable to the other variations, embodiments, or constructions unless explicitly stated otherwise.

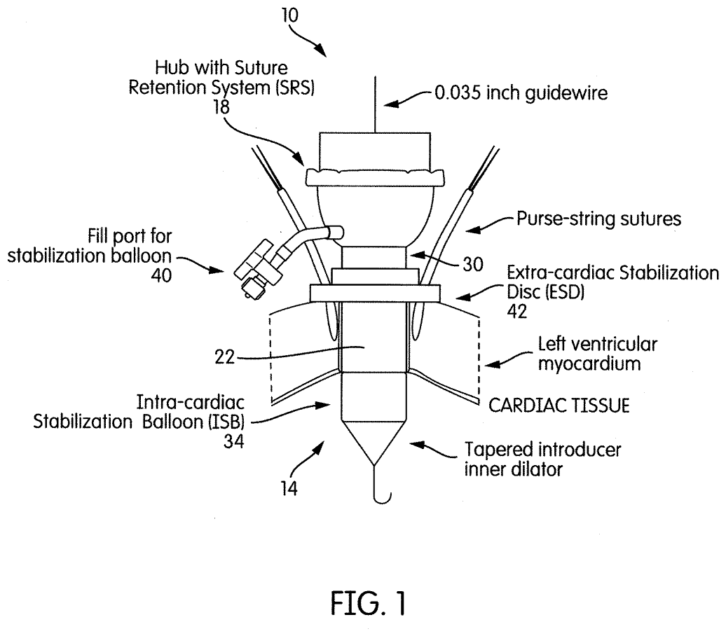

FIGS. 1-3 illustrate an intra-cardiac introducer device 10 for use when performing transventricular “beating heart” suture mediated repair of mitral and aortic valves. The intra-cardiac introducer device 10 includes an introducer sheath 14, a hub 18, an intra-cardiac stabilization balloon 34, an external stabilization disc 42, a valve system 54, and a suture retention system 74. In one construction, the introducer sheath 14 is a short, large caliber vascular sheath (e.g., 16, 18, 20, 22, 24, 26, 28, 30, 32, 34, and 36 French).

The introducer sheath 14 is a generally tubular structure or shaft 22 having a distal end 26 and a proximal end 30. The introducer sheath 14 is configured to be delivered through cardiac tissue over a short stiff guide wire (e.g., 0.035 inch). The distal end 26 of the introducer sheath 14 is tapered for insertion through and dilation of the cardiac tissue.

The introducer sheath 14 also includes an intra-cardiac stabilization balloon 34 connected to an outer surface 38 of the tubular structure 22. The balloon 34 is an integrated, soft, compliant balloon in communication with an insufflation port 40 to be inflated with fluid (e.g., saline). In one construction, the balloon 34 comprises a low durometer material resulting in an ultra-compliant balloon to improve sheath maneuverability and reduce the risk of balloon rupture. The balloon 34 is configured to be in a deflated state while the introducer sheath 14 is being inserted into the left or right ventricle and in an inflated state after the introducer sheath 14 is properly positioned within the intraventricular cavity. The inflated state of the balloon 34 prevents removal of the device 10 from the left or right ventricle, provides intraventricular stabilization of the introducer, and minimizes hemostasis.

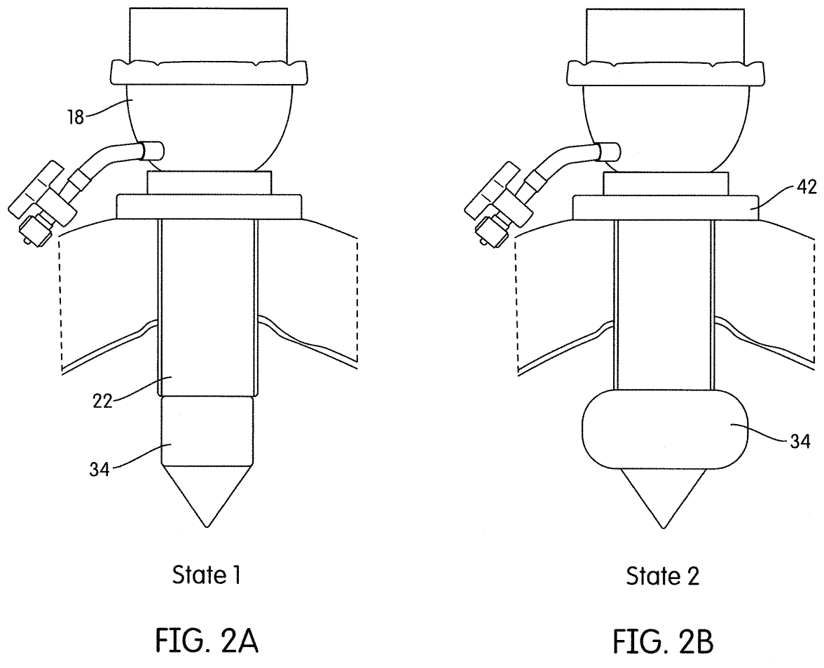

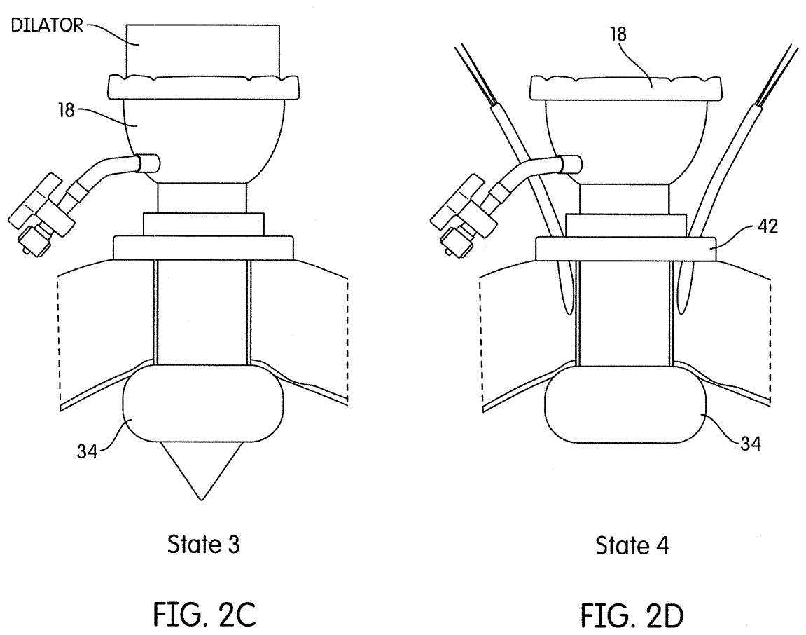

With more particularity and with reference to FIGS. 2A-D, the intra-cardiac stabilization balloon 34 when in a deflated first state, is located at the distal end 26 of the sheath 14 with minimal outer diameter profile change compared to the outer diameter of the sheath shaft 22. When inflated to the second state, with fluid (e.g., saline), the balloon 34 expands radially from the sheath 14, which is located within the ventricular cavity (FIG. 2B). The radial expansion ranges from about 3 mm to about 12 mm depending on the volume of fluid infused into the inflation port and into the balloon 34. When inflated, a distal end of the balloon 34 is located at the distal end 26 of the sheath shaft 22. When the balloon 34 is inflated with fluid (e.g., saline), the sheath 14 is retracted to contact the inner myocardial surface of the ventricle as shown at state 3 (FIG. 2C).

An inner dilator is then removed from the sheath shaft 22, and the trans myocardial purse-strings sutures are then secured to the extra-cardiac stabilization disc 42 as shown at state 4 (FIG. 2D). The intra-cardiac stabilization balloon 34 and the extra-cardiac stabilization disc 42 work together to maintain sheath stability throughout the transventricular surgery.

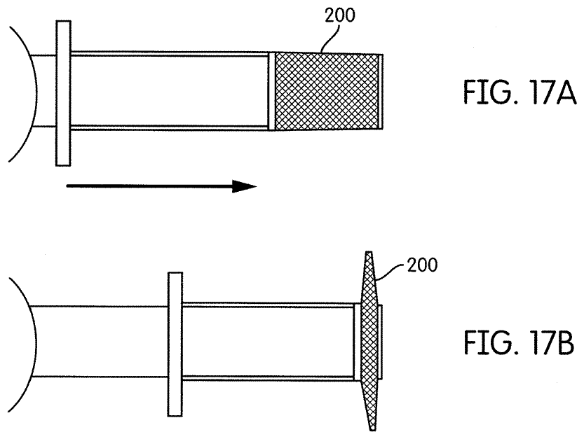

In an alternative embodiment, rather than an intra-cardiac stabilization balloon 34, a compliant coated nitinol disc 200 may be employed for internal stabilization as illustrated in FIGS. 17A-B. When the nitinol disc 200 is slidingly actuated, it expands to form an intracardiac stabilization platform with similar stabilization features as described above with respect to the balloon 34.

The hub 18 is coupled to the proximal end 30 of the introducer sheath 14. The hub 18 is generally “bowl-shaped” and includes a gradually increasing diameter from the coupling at the distal end 26 to the hub's proximal end. The hub 18 includes a cavity 50 and is in fluid communication with the tubular structure 22 to receive blood exiting the heart due to the surgical procedure.

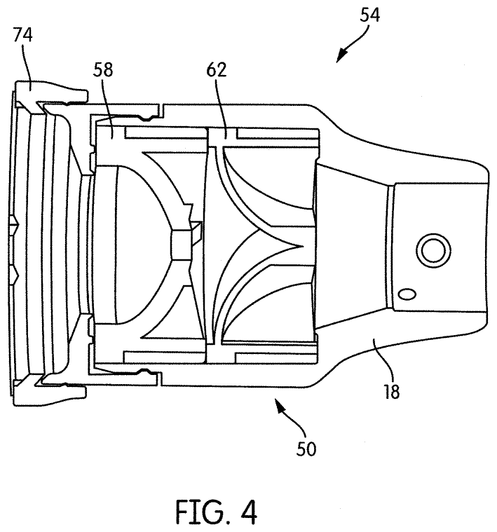

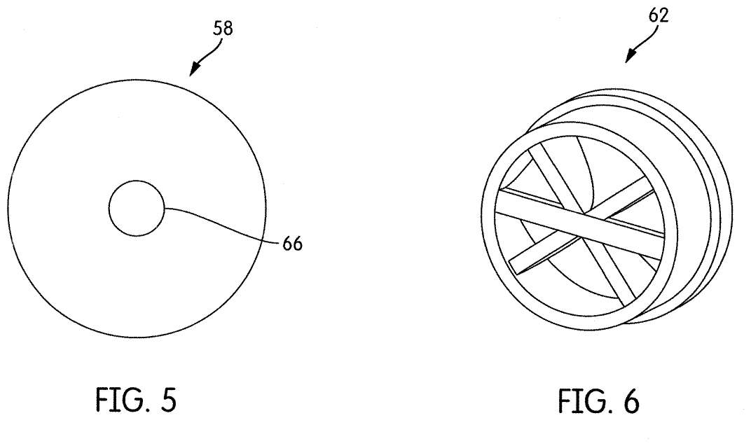

The hub 18 includes a dual valve system 54 to facilitate easy passage of the introducer sheath 14 and provide sufficient hemostasis during the procedure. With reference to FIGS. 4-6, the valve system 54 includes a first valve 58 and a second valve 62. The first valve 58 is at least partially positioned within the cavity 50 of the hub 18. A portion of the first valve 58 is visible at the proximal end of the hub 18. The first valve 58 is circular in shape having a perimeter generally consistent with the perimeter of the proximal end of the cavity 50. The first valve 58 includes a central aperture 66 that is configured to receive an intraventricular/endovascular device 70 intended for use with a transventricular method and provide hemostasis while the device 70 is within the introducer sheath 14. The second valve 62 is positioned within the cavity 50 of the hub 18 and includes a double, reinforced duckbill design to provide hemostasis following removal of the device 70 and with exiting sutures crossing the hub valves 58, 62.

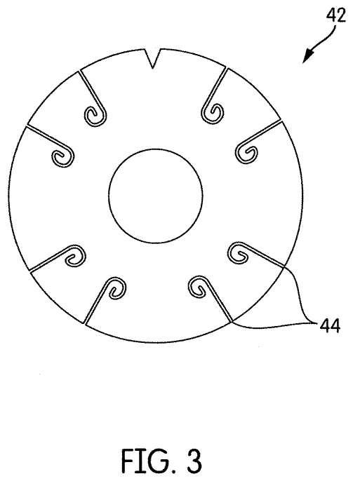

The extra-cardiac stabilization disc 42, illustrated in FIG. 3, is positioned circumferentially on the tubular structure 22 forward of the hub 18 and along the outer surface of the left or right ventricle. The extra-cardiac stabilization disc 42 is slidingly coupled to the exterior surface of the tubular structure 22 and can be comprised of silicone, silastic, or other suitable materials. The extra-cardiac stabilization disc 42 includes a plurality of slots 44 for receiving purse-string sutures via tourniquets 46 to further enhance stabilization of the sheath 14.

The extra-cardiac stabilization disc 42 includes two components, functioning as one unit. The larger diameter component contacts the epicardial surface of the ventricle and includes the slots 44 for inserting and securing the trans myocardial purse-string sutures. The smaller, and more proximal, diameter component limits proximal migration of the extra-cardiac stabilization disc 42. The extra-cardiac stabilization disc 42 and intracardiac balloon 34 work together to provide a stable transventricular introducer sheath while still maintaining maneuverability for medical device insertion and removal.

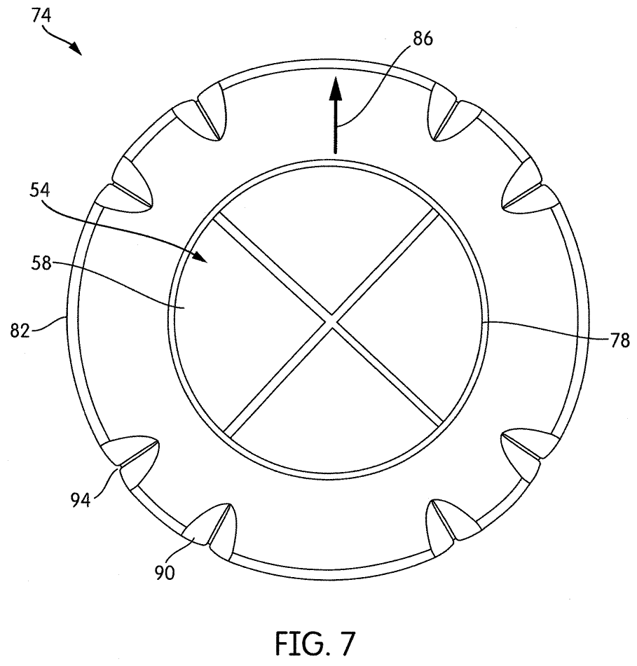

With reference to FIG. 7, the device 10 also includes a suture retention system 74. The removable suture retention system 74 is coupled to the hub 18 via a mechanism that allows a bottom surface to snap onto or mate with the hub 18. The suture retention system 74 includes a central aperture 78 configured to align with the proximal end of the hub 18. During suture mediated transventricular structural heart surgery, there are anywhere from 2-8 or more additional sutures attached to the mitral valve leaflet exiting the left ventriculotomy. There is a need for organization of the sutures, as well as retention during the surgery to limit the number of additional instruments in the narrow surgical window.

The suture retention system 74 is generally circular-shaped and includes a body 82 having one or more orientation markers 86, e.g., 12 o'clock, 3 o'clock, 6 o'clock, 8 o'clock, and 9 o'clock, but more or less than these example markers are also contemplated. The orientation markers 86 generally function to orient the position of the sutures relative to the heart and provide an ordering system to ensure correct placement of the sutures. The body 82 also includes a plurality of recesses 90 and slots 94 formed in the body 82 to safely retain the sutures without damaging the suture material. The slots 94 may include a silicone filler for improved suture retention. By color marking the tail ends of the long sutures, the position of the sutures can be organized in a manner that facilitates the most efficient and accurate final placement. The exiting sutures can be secured in the slots 94. The suture retention system 74 maintains proper suture organization and reduces the chance of suture disruption during re-entry of the device 70 into the introducer sheath 14 for additional suture placement(s).

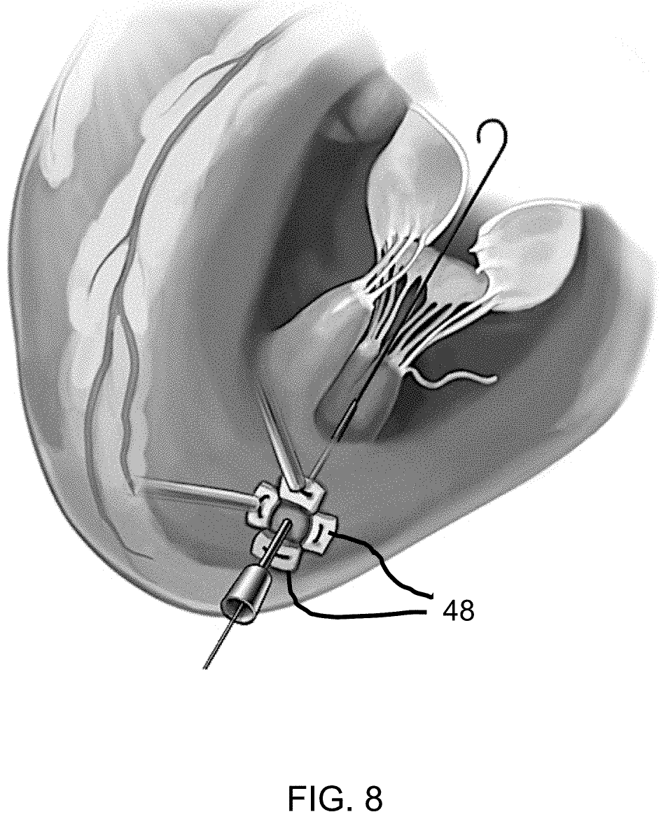

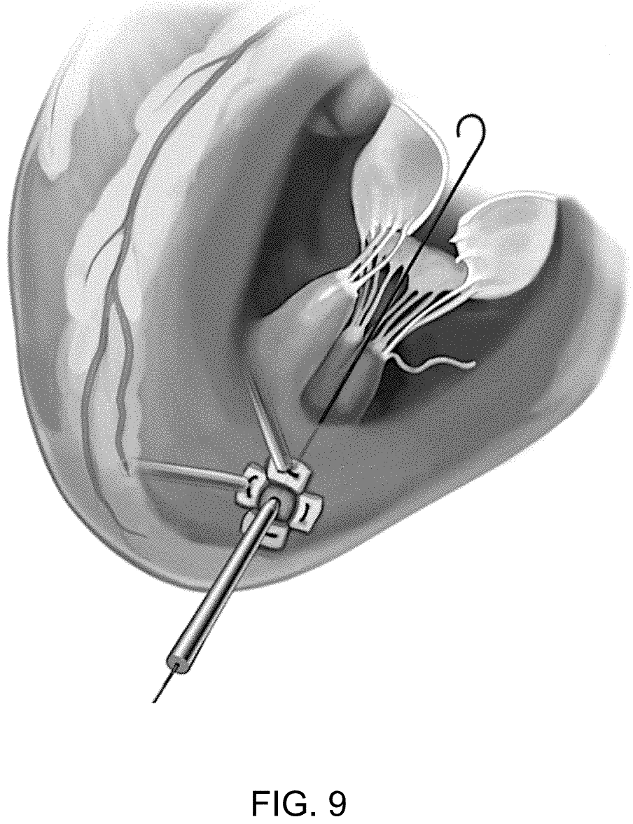

In operation, initially, the surgeon places purse-strings pledget sutures into the cardiac tissue (e.g., left ventricular apical myocardium) (surgical purse-string) for future closure of the ventriculotomy, followed by a puncture within the center of the purse-string closure using an 18 gauge (or other appropriate size) needle, followed by guide wire advancement into the left atrium, ascending aorta, right atrium, or main pulmonary artery depending on the transventricular surgery being performed. (See FIG. 8) The myocardial tract is initially dilated with vascular dilators to dilate the ventricular opening to accommodate the introducer sheath. The intraventricular introducer sheath 10 is then positioned into the ventricular cavity with the inner dilator 14 delivered over the guidewire and into the left or right ventricle. (See FIG. 9)

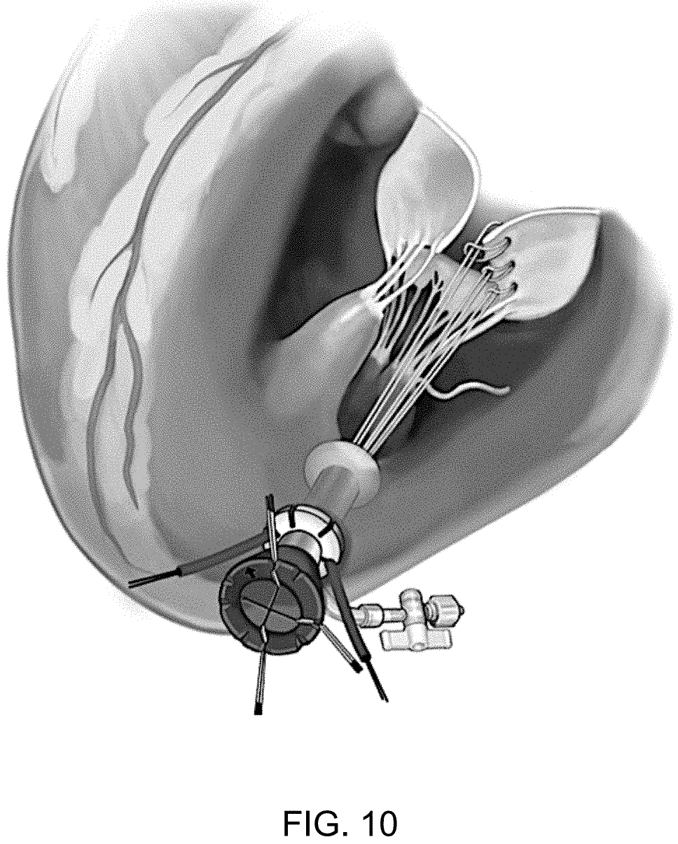

The introducer sheath 14 includes linear markings indicating whether proper intraventricular placement has been achieved using trans esophageal echocardiographic and/or fluoroscopic guidance. After proper placement of the introducer sheath 14, the balloon 34 is inflated with fluid (e.g., saline). Proper placement assures that there is no inadvertent expansion of the balloon 34 within the myocardium leading to injury. After the balloon 34 is prepped and filled with fluid, the introducer sheath 14 is gently pulled back to provide hemostasis at the inner margin of the ventriculotomy. Then, the external stabilization disc 42 is slid along the tubular structure 22 and positioned on top of felt pledgets 48 (see FIGS. 8-10) to stabilize the position of the introducer sheath 14 from the balloon 34 to the external stabilization disc 42. The previously placed purse-string sutures 46 are inserted into the slots 44 in the external stabilization disc 42, that when affixed to the disc 42 via plastic tourniquets afford improved stability and security of the introducer sheath 14. (See FIGS. 9-10)

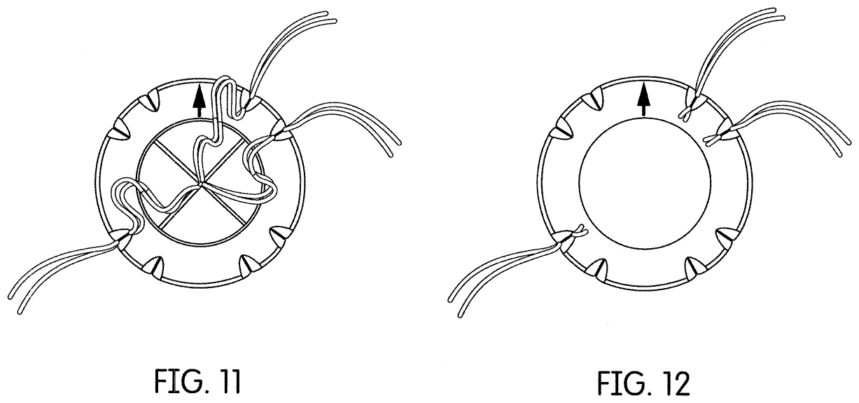

Once the surgeon has completed placement of the sutures inside the heart, for mitral valve repair, for example, each suture is positioned in the center of the valve system 54, and then positioned in one of the slots 94 of the suture retention system 74. The sutures are retained at the tips only with color marking on both sides of the slots 94. This is performed for all of the retained sutures. (See FIGS. 10-11) Next, the sutures are cut leaving color markings on the long end of the suture, as well as the short end which is still retained in the slots 94 on the hub 18. (See FIG. 12) Next, in preparation for closure of the ventriculotomy, the tourniquets are removed from the purse-string sutures encircling the ventriculotomy, the sutures are then removed from the external stabilization disc 42. The balloon 34 on the sheath is fully deflated, confirmed by trans esophageal echocardiography and/or fluoroscopy, and the sheath is then removed leaving the color coded sutures exiting the ventriculotomy. The purse-strings sutures are tightened to maintain hemostasis following sheath removal.

The color-coded valve repair sutures are then secured to a felt pledget 48 as directed by the instructions for use by the manufacturer of the device in use for the transventricular suture mediated structural heart surgery. The suture retention system 74, with all color-coded retained sutures from surgery, is then detached from the hub 18 of the introducer sheath 14 and then utilized as a reference for the proper suture placement (medial vs. lateral for example) in the felt pledget 48 for surgical procedure completion.

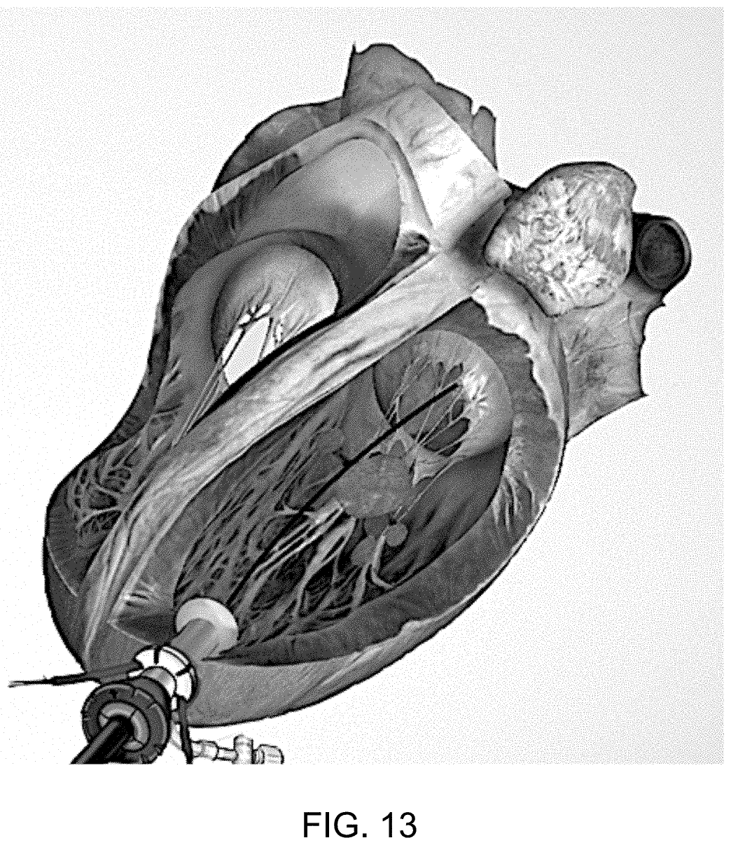

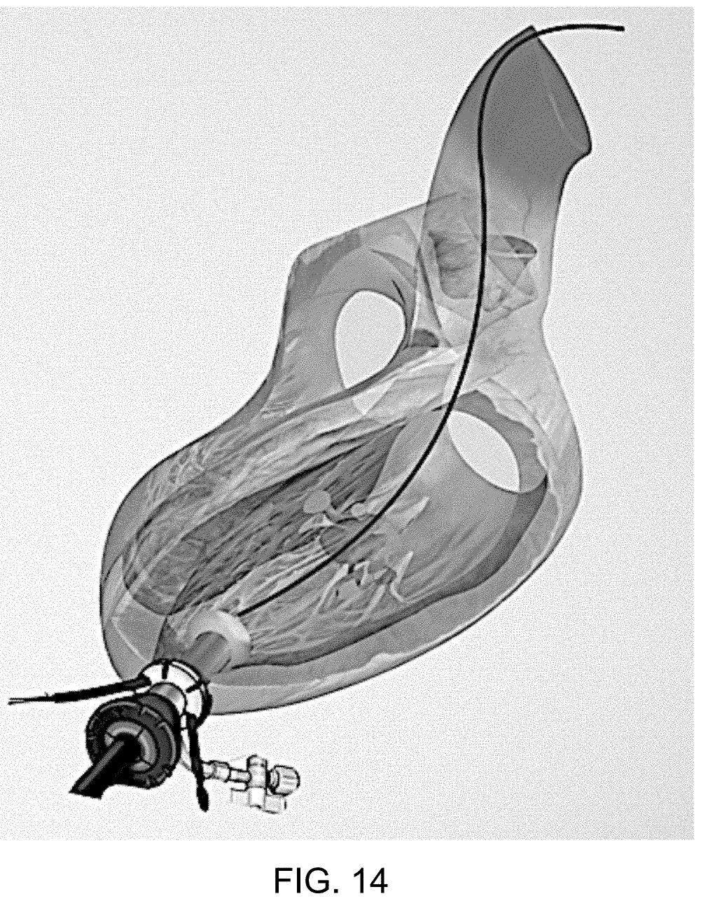

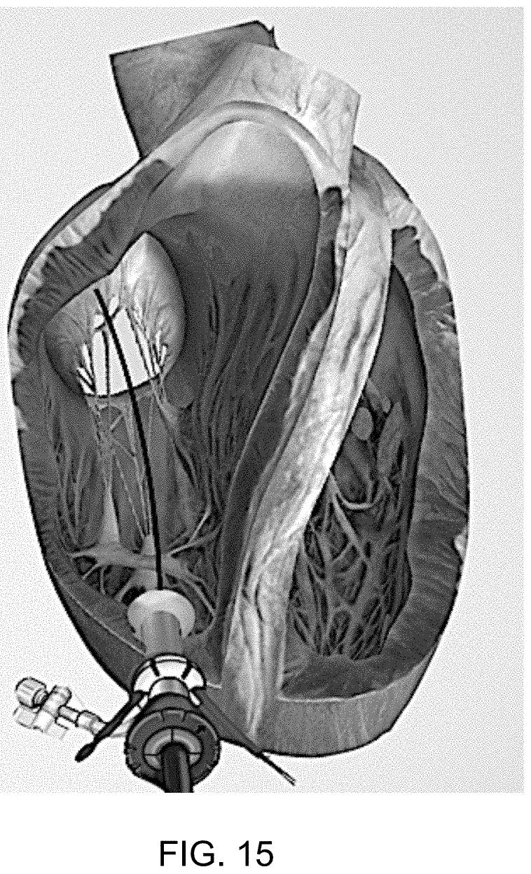

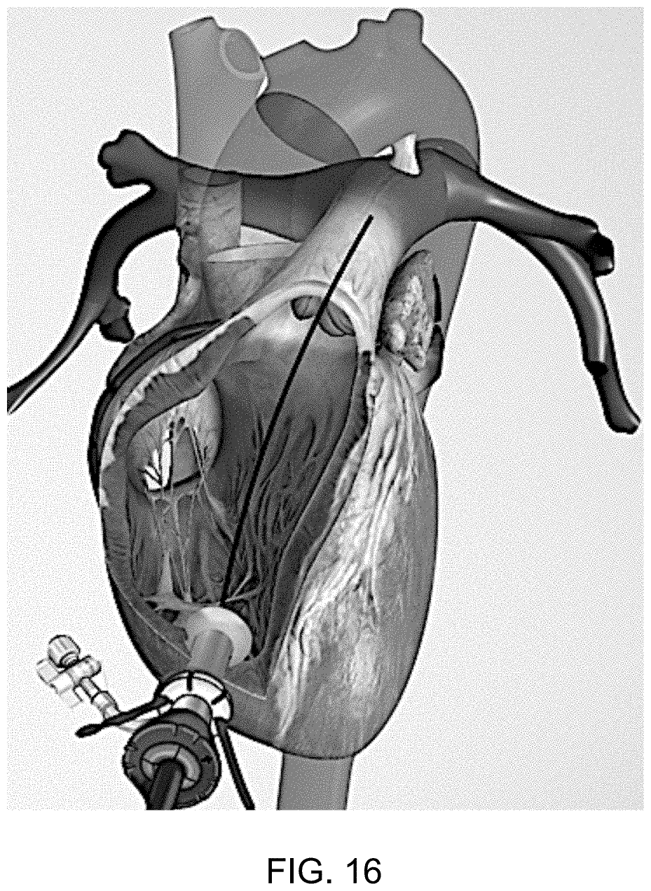

FIGS. 13-16 illustrate additional renderings of embodiments of intra-cardiac introducer device 10 being utilized to access the mitral valve, aortic valve, tricuspid valve and pulmonic artery, respectively.

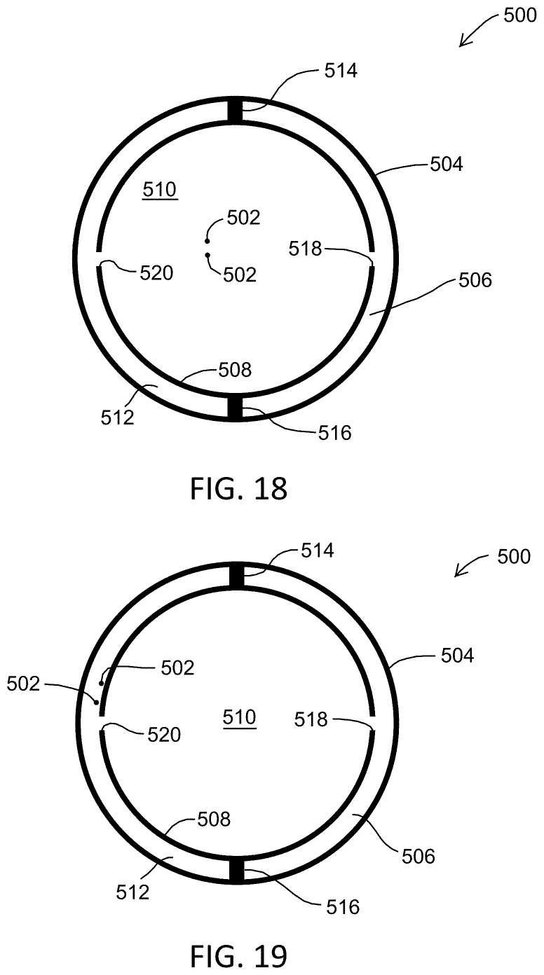

FIGS. 18 and 19 illustrate another construction of a tubular structure 500 that could be used in place of the tubular structure 22 to enhance the handling and organization of the sutures 502. The tubular structure 500 or shaft of the sheath 14 defines two lumens. More specifically, the tubular structure 500 includes an outer wall 504 or tube that defines a first lumen 506 or cylindrical space. An inner wall 508 or tube is disposed within the first lumen 506 to define a second lumen 510 therein. The outer wall 504 and the inner wall 508 are preferably arranged along a common axis and define an annular space 512 therebetween. Each of the inner wall 508 and the outer wall 504 extends the full length of the tubular structure 500 between a proximal end 30 and a distal end 26. However, other arrangements may include an inner wall 508 that is shorter than the outer wall 504 in one or both directions.

A first rib 514 extends between the inner wall 508 and the outer wall 504 to fixedly attach the outer wall 504 and the inner wall 508 to one another. In the illustrated construction, the first rib 514 extends the full length of the tubular structure 500. However, other constructions could include a shorter first rib 514 or a first rib 514 that is broken or discontinuous along the length of the tubular structure 500. A second rib 516 is positioned circumferentially opposite the first rib 514 between the outer wall 504 and the inner wall 508. Generally, the second rib 516 is the same as the first rib 514. However, the first rib 514 and the second rib 516 could be different if desired. In addition, while FIGS. 18 and 19 illustrate the first rib 514 and the second rib 516 spaced 180 degrees apart, other constructions may include different spacings between the first rib 514 and the second rib 516. In addition, other constructions could include a single rib, or could include three or more ribs that could be spaced circumferentially from one another at equal angles or different angles. For example, three ribs spaced 120 degrees apart could be employed. It should be understood that the ribs 514, 516 cooperate to break the annular space 512 into multiple separate spaces with the number of spaces being generally equal to the number of ribs (i.e., two ribs produce two separate spaces).

While FIGS. 18 and 19 illustrate the inner wall 508, the outer wall 504, the first rib 514, and the second rib 516 as one continuous and inseparable piece, other arrangements could form one or more of the first rib 514, the second rib 516, the inner wall 508, or the outer wall 504 as separate pieces that are permanently or removably attached to one another.

A first slot 518 is formed through the inner wall 508 to provide access to the annular space 512 between the inner wall 508 and the outer wall 504. In the illustrated construction, the first slot 518 is formed 90 degrees circumferentially from the first rib 514 and the second rib 516 with other positions with respect to the first rib 514 and the second rib 516 being possible. The first slot 518 extends the full length of the inner wall 508. In addition, while the first slot 518 is illustrated as being parallel to the longitudinal axis of the tubular section 500, other constructions could include a first slot 518 that is obliquely angled with respect to the longitudinal axis.

A second slot 520 that is substantially the same as the first slot 518 is formed at a circumferential position that is 180 degrees from the first slot 518. Of course, there is no requirement that the first slot 518 and the second slot 520 be identical or that they be positioned 180 degrees apart. Each of the first slot 518 and the second slot 520 preferably provide access to different spaces in the annular space 512. Thus, other constructions that include additional ribs may include additional slots to assure that each space defined by the ribs is accessible. For example, a construction that includes three ribs would define three separate spaces within the annular space 512. Three slots could be provided to provide access to each of those three spaces.

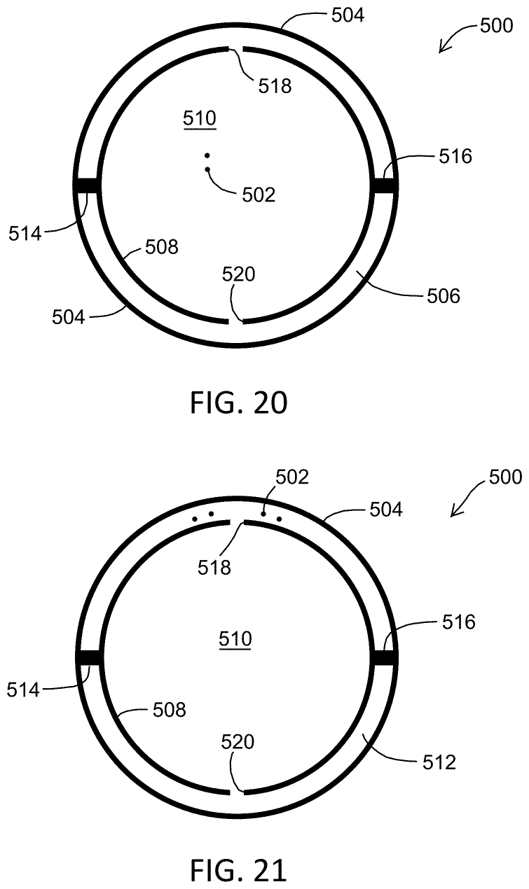

In addition, as illustrated in FIGS. 20 and 21, the orientation of the first slot 518 and the second slot 520 can be varied. Specifically, in the arrangement of FIGS. 20 and 21, the tubular structure 500 is rotated ninety degrees when compared to the arrangement illustrated in FIGS. 18 and 19. It should be clear that any orientation of the tubular structure desired could be employed.

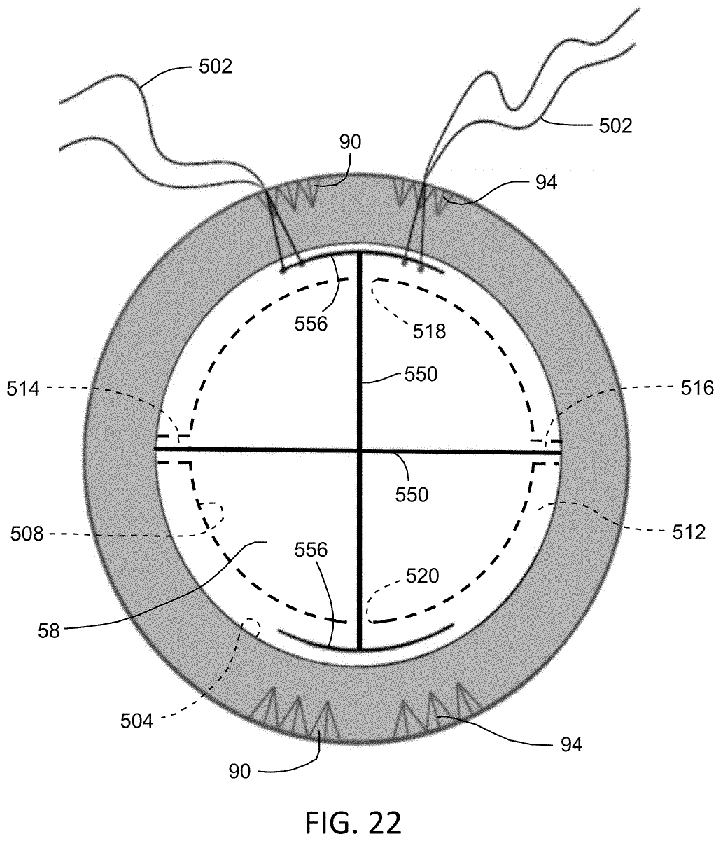

FIG. 22 is similar to FIG. 11 and illustrates a top view of the suture retention system connected to the hemostatic hub using the tubular structure 500 illustrated in FIGS. 18-21. The opening aperture of the second valve 62 (duckbill valve) is oriented such that the longitudinal axis of the opening is in line with at least one but preferably both of the first slot 518 and the second slot 520 in the inner wall 508 of the tubular structure 500 of the sheath 14. The outer valve or first valve 58 of the sheath 14 is configured with two or more slits 550 and one of those slits 550 is oriented parallel to the opening axis of the second valve 62 such that it aligns with one or both of the first slot 518 and the second slot 520.

In the arrangement illustrated in FIG. 22, the first valve 58 includes two longitudinal slits 550 with at least one oriented with the first slot 518 and the second slot 520 in the inner wall 508 of the tubular structure 500. At least one of these slits 550 extends into and intersects one or more curvilinear slits 556 arranged to extend in an arc positioned over the annular space 512 formed between the outer wall 504 and the inner wall 508.

During transventricular, suture mediated structural heart surgery, the sutures 502 are directed along the lateral slits 550 in the valves 58, 62 and the curvilinear slits 556 to position the sutures 502 in one of the annular spaces 512 between the inner wall 508 and the outer wall 504 via one of the first slot 518 and the second slot 520 in the inner wall 508 of the tubular structure 500. The sutures 502 are then guided along the curvilinear portion of the valve 58 to seat the sutures 502 between the inner wall 508 and the outer wall 504 of the tubular structure 500 to limit the interaction between already deployed sutures 502 with the suture delivery system or tool.

The sutures 502 are secured between the inner wall 508 and the outer wall 504 of the tubular structure 500 for the remainder of the surgery, with subsequently deployed sutures 502 secured in a similar manner along the right or left side of the tubular structure 500. Once the surgery is near completion, the labeled sutures 502 are left in place with gentle retraction of the sheath 14, removal of the sheath 14 from the ventriculotomy, and closure of the ventriculotomy site by tightening of the purse-string sutures. The deployed sutures 502 are then tensioned and secured to the ventriculotomy site.

The intra-cardiac introducer device 10 provides cardiothoracic surgeons a stable transventricular specific introducer sheath system making the surgery safer for patients by minimizing blood loss, enhancing device delivery making the surgery more precise and efficient, and providing a platform for fewer surgical instruments in the small surgical window.

Although exemplary embodiments of the invention have been described in detail with reference to certain preferred embodiments, those skilled in the art will understand that variations and modifications exist within the scope and spirit of one or more independent aspects of the invention as described and recited in the following claims.

What is claimed is:

1. An intra-cardiac introducer device comprising:
a sheath including a tubular portion having an inner wall and an outer wall spaced apart from the inner wall to define an annular space, the sheath defining a distal end and a proximal end and arranged to penetrate cardiac tissue;
a hub connected to the proximal end of the sheath;
an intracardiac stabilization balloon coupled to the distal end of the sheath; and
an external stabilization disc slidingly positioned along the sheath;
wherein the cardiac tissue is sandwiched between the intracardiac stabilization balloon and the external stabilization disc to secure the position of the sheath in the cardiac tissue.

2. The device of claim 1, further comprising a valve system coupled to the hub to provide hemostasis during transventricular surgery.

3. The device of claim 2, wherein the valve system includes a first valve coupled to a proximal end of the hub and a second valve positioned within a cavity of the hub, and wherein the first valve includes a curvilinear slit positioned over the annular space.

4. The device of claim 1, wherein the intracardiac stabilization balloon transitions between an inflated state and a deflated state.

5. The device of claim 1, wherein the balloon is coupled to the outer wall of the tubular portion.

6. The device of claim 1, wherein the stabilization disc includes a plurality of slots configured to receive sutures.

7. The device of claim 6, wherein at least a portion of each suture is selectively positionable in the annular space.

8. The device of claim 1, wherein the inner wall defines a slot that extends from the distal end to the proximal end to facilitate movement of each of a plurality of sutures into and out of the annular space.

9. The device of claim 1, further comprising a first rib and a second rib extending from the inner wall to the outer wall to fixedly attach the inner wall to the outer wall.

10. The device of claim 1, wherein the inner wall includes a hollow interior and is configured to receive a guidewire with an inner dilator in place for device insertion.

11. An intra-cardiac introducer device comprising:
a sheath including a tubular portion having an inner wall and an outer wall each extending from a distal end to a proximal end, the inner wall and the outer wall arranged concentrically to define an annular space;
a hub connected to the proximal end of the sheath; and
a suture retention system removably coupled to the hub, the suture retention system including a body, a plurality of slots on the body configured to receive sutures, and an orientation marker configured to identify a position of the sutures relative to a patient's heart, wherein the sutures are selectively disposed at least partially within the annular space.

12. The device of claim 11, further comprising a valve system coupled to the hub and configured to provide hemostasis during transventricular surgery.

13. The device of claim 12, wherein the valve system comprises a first valve coupled to a proximal end of the hub and a second valve positioned within a cavity of the hub, and wherein the first valve includes a curvilinear slit positioned over the annular space.

14. The device of claim 11, further comprising a balloon coupled to the sheath, the balloon operable to transition between an inflated state and a deflated state.

15. The device of claim 14, wherein the balloon is coupled to the outer wall of the sheath at the distal end.

16. The device of claim 14, further comprising a stabilization disc coupled to the outer wall, the stabilization disc positioned between the heart and the hub to stabilize the sheath when the balloon is in the inflated state.

17. The device of claim 16, wherein the stabilization disc includes a plurality of slots configured to receive purse-string sutures.

18. The device of claim 17, wherein at least a portion of each purse-string suture is selectively positionable in the sheath space.

19. The device of claim 11, wherein the inner wall defines a slot that extends from the distal end to the proximal end to facilitate movement of each of a plurality of sutures into and out of the annular space.

20. The device of claim 11, further comprising a first rib and a second rib extending from the inner wall to the outer wall to fixedly attach the inner wall to the outer wall.
